(19) United States
(12) Patent Application Publication
(10) Pub. No.: US 2013/0317314 A1
(43) Pub. Date: Nov. 28, 2013

(54) Vibrating Comedo Extractor with Light

(71) Applicant: David Patrick Lampson, Grass Valley, CA (US)

(72) Inventor: David Patrick Lampson, Grass Valley, CA (US)

(21) Appl. No.: 13/954,066

(22) Filed: Jul. 30, 2013

Related U.S. Application Data
(60) Provisional application No. 61/677,344, filed on Jul. 30, 2012.

(51) Int. Cl. A61B 17/50; A61B 1/06

(52) U.S. Cl. CPC A61B 17/50; A61B 1/06
USPC 600/249

(57) ABSTRACT

A vibrating comedo extractor with light to aid in immediate removal of blemishes to allow healthier and better-looking skin. The vibrating comedo extractor with light generally includes a housing having an extractor extending from a frontal opening thereof. A motor is positioned within the housing for imparting vibrational force to the extractor. An illumination device is positioned adjacent to the extractor to aid in lighting the area being treated. By activating the motor and light using a switch on the housing, the present invention may be utilized to efficiently, accurately and safely remove various blemishes such as comedones from the skin.

### CROSS REFERENCE TO RELATED APPLICATIONS

I hereby claim benefit under Title 35, United States Code, Section 119(e) of U.S. provisional patent application Ser. No. 61/677,344 filed Jul. 30, 2012. The 61/677,344 application is currently pending. The 61/677,344 application is hereby incorporated by reference into this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to a comedo extractor and more specifically it relates to a vibrating comedo extractor with light to aid in immediate removal of blemishes to allow healthier and better-looking skin.

2. Description of the Related Art

Any discussion of the related art throughout the specification should in no way be considered as an admission that such related art is widely known or forms part of common general knowledge in the field.

Extractors for removing comedones are known in the art for removing various blemishes from the skin. However, prior art comedo extractors suffer from a number of shortcomings. The prior art devices are often difficult to use. They are thin and hard to hold securely, which can result in misplacement of the tool and necessitate multiple attempts to remove the blemish, possibly causing redness, bruising, irritation, pain and scarring.

Prior art devices also lack any illumination, which either necessitates a more difficult extraction or the use of a separate illumination device to light the area from which the blemish is being removed. Finally, prior art devices do not include any vibrational features, which result in additional force and manipulation being required to adequately remove a blemish.

Because of the inherent problems with the related art, there is a need for a new and improved vibrating comedo extractor with light to aid in immediate removal of blemishes to allow healthier and better-looking skin.

### BRIEF SUMMARY OF THE INVENTION

The invention generally relates to a comedo extractor which includes a housing having an extractor extending from a frontal opening thereof. A motor is positioned within the housing for imparting vibrational force to the extractor. An illumination device is positioned adjacent to the extractor to aid in lighting the area being treated. By activating the motor and light using a switch on the housing, the present invention may be utilized to efficiently, accurately and safely remove various blemishes such as comedones from the skin.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention.

FIG. 2 is an upper perspective view illustrating usage of the present invention to remove a comedo.

FIG. 3a is a first side view of the present invention.

FIG. 3b is a second side view of the present invention illustrating depression of the switch and vibration of the extractor.

FIG. 4 is a top view of the present invention.

FIG. 5 is a frontal view of the present invention.

FIG. 6 is a side sectional view of the present invention.

FIG. 7 is an upper perspective view of the present invention utilizing an alternate extractor tip.

FIG. 8 is an upper perspective view of the present invention utilizing an alternate extractor tip.

FIGS. 9a-9e are upper perspective views of various different extractor tips used with the present invention.

### DETAILED DESCRIPTION OF THE INVENTION

### A. Overview.

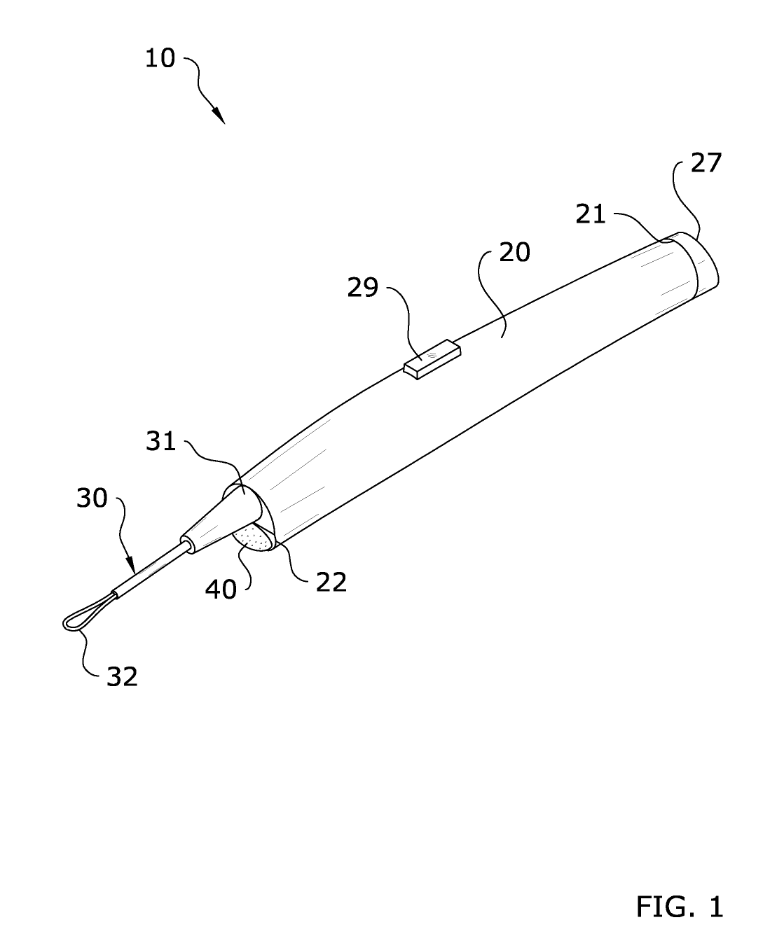

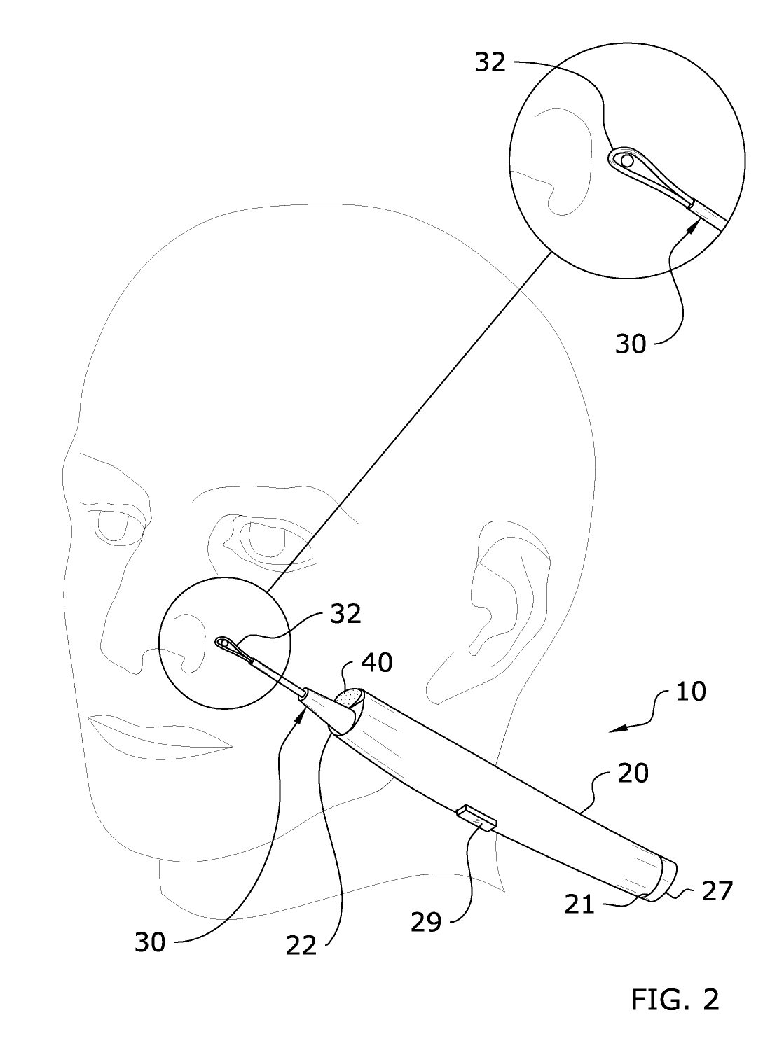

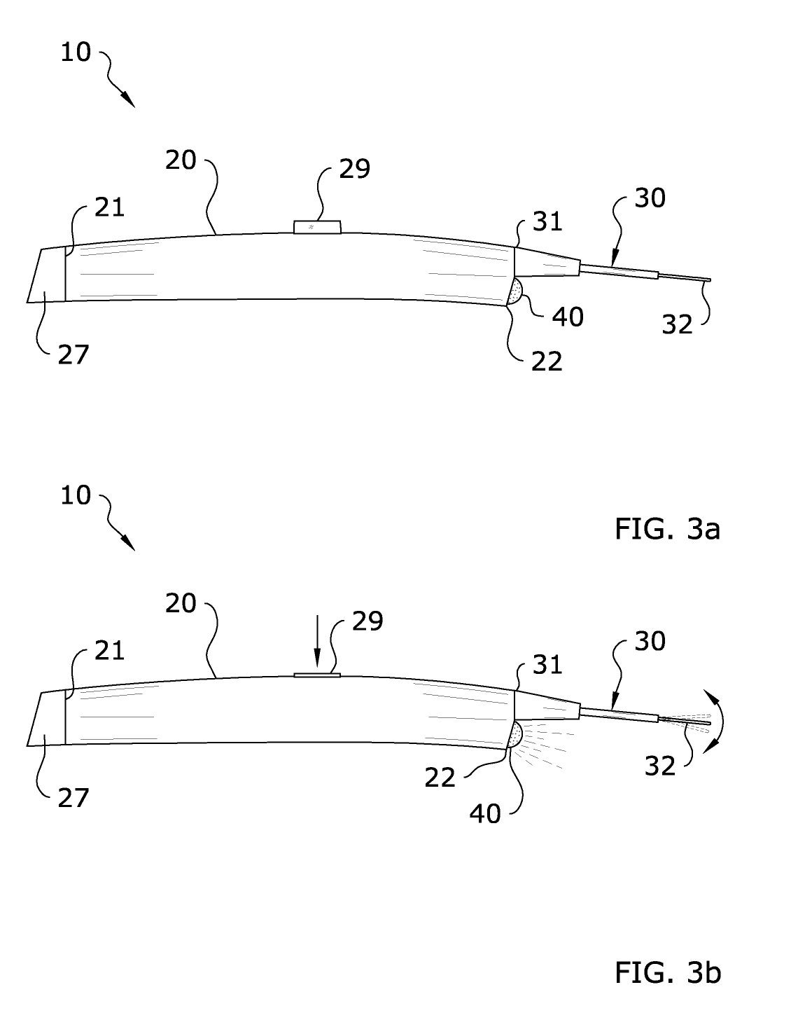

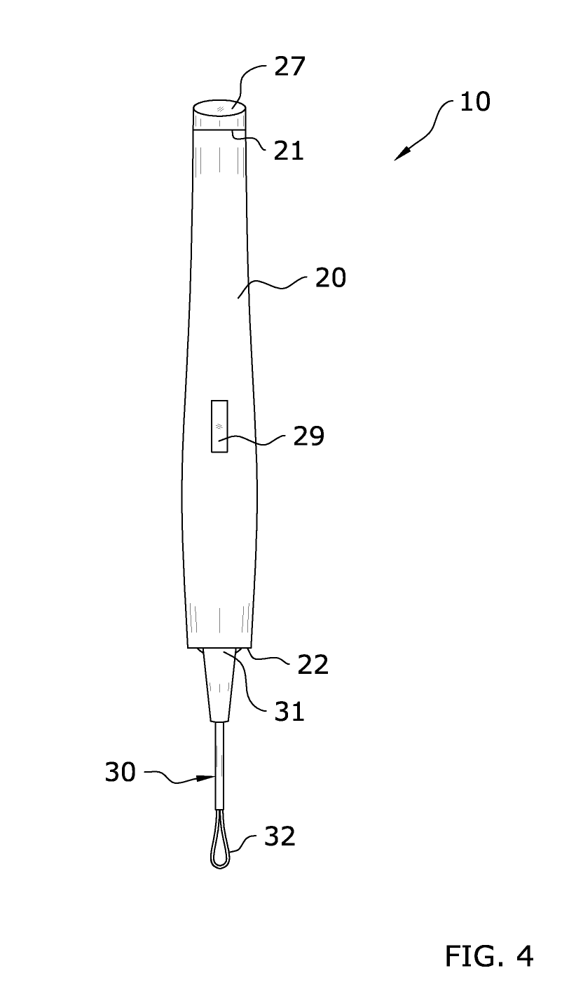

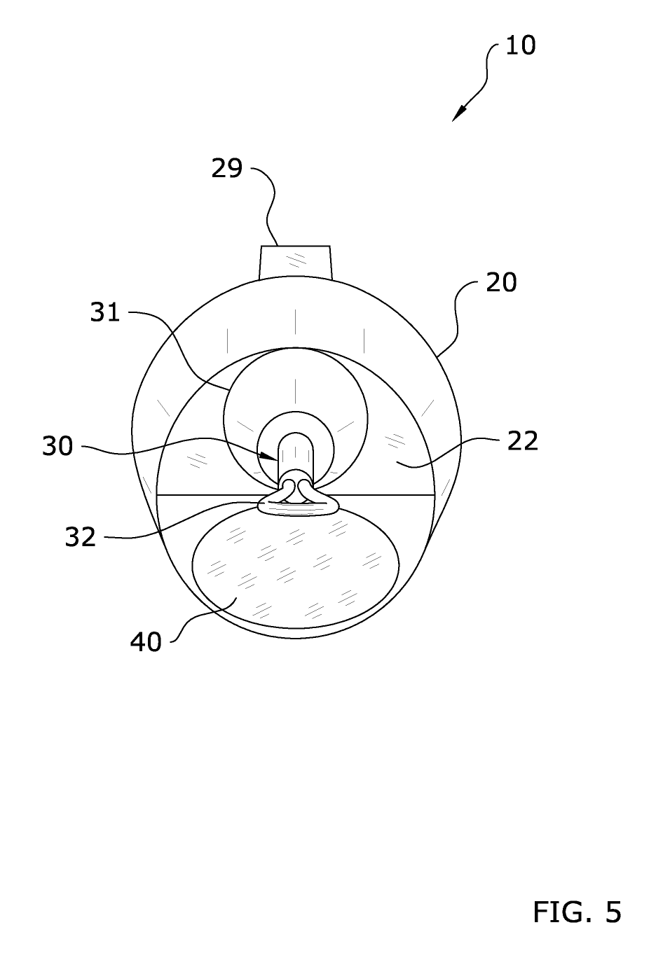

Turning now descriptively to the drawings, in which similar reference characters denote similar elements throughout the several views, FIGS. 1 through 7 illustrate a vibrating comedo extractor with light 10, which comprises a housing 20 having an extractor 30 extending from a frontal opening 23 thereof. A motor 54 is positioned within the housing 20 for imparting vibrational force to the extractor 30. An illumination device 40 is positioned adjacent to the extractor 30 to aid in lighting the area being treated. By activating the motor 54 and light 40 using a switch 29 on the housing 20, the present invention may be utilized to efficiently, accurately and safely remove various blemishes such as comedones from the skin.

### B. Housing.

As shown throughout the figures, the present invention includes a housing 20 which stores the various components of the present invention. The housing 20 may be comprised of various configurations, shapes, and sizes, and thus should not be construed as being limited in any of these respects to the exemplary embodiments shown in the figures. The housing 20 includes a first end 21, which acts as a gripping portion/handle of the present invention, and a second end 22, from which the extractor 30 of the present invention extends. The handle portion 21 of the housing 20 may be ergonomically designed for improved comfort.

The first end 21 of the housing 20 includes a battery receiver 25 which is adapted to removably receive a power source 50 such as a battery 50 therein. As shown throughout the figures, the battery receiver 25 includes a threaded portion 26 adapted to removably receive a threaded cap 27 therein to enclose the power source 50 within the housing 20. The second end 22 of the housing 20 includes a frontal opening 23 through which the extractor 30 extends as shown in FIG. 1.

The housing 20 includes an interior portion 28 which stores the various components of the present invention, such as the power source 50, circuit board 52, motor 54 and conduits 56. The interior portion 28 will preferably be at least partially hollow to accommodate these components.

The housing 20 will also generally include a switch 29 which generally at least partially extends out of its exterior surface. Various types of switches 29 may be utilized, such as a button configuration as shown in the figures. In some embodiments, the switch 29 may be flush with the exterior surface of the housing 20. In other embodiments as shown in the figures, the switch 29 extends partially out from the housing 20. The switch 29 may be positioned at various locations on the housing 20 and thus should not be construed as limited to the positioning shown in the figures.

In some embodiments, the housing 20 may also include such add-ons as a reservoir for liquid, a spray jet, anti-microbial sponge, a brush tip, pull-out disposable wipes, suction, a micro facial steamer, and/or a styptic pencil.

### C. Extractor.

As shown throughout the figures, an extractor 30 extends out of the front opening 23 of the housing 20. The extractor 30 generally comprises a first end 31 and a second end 32. The first end 31 of the extractor 30 includes a connector portion 33 which is adapted to engage with a corresponding extractor holder 35 positioned within the frontal opening 23 of the housing 20. The second end 32 of the extractor 30 comprises an extractor tip which is utilized to aid in removal of comedones or other blemishes on the skin.

The extractor 30 may be fixedly or removably secured within the extractor holder 35. In some embodiments, the extractor holder 35 may be omitted and the extractor 30 fixedly or removably secured directly within the frontal opening 23 of the housing 20. In other embodiments, the extractor 30 may be removably secured within the housing 20 by such methods as magnetic, friction fit, knurled fit, threaded engagement, clamps, shank and collet, slidable securing, pivoting, or push-pin.

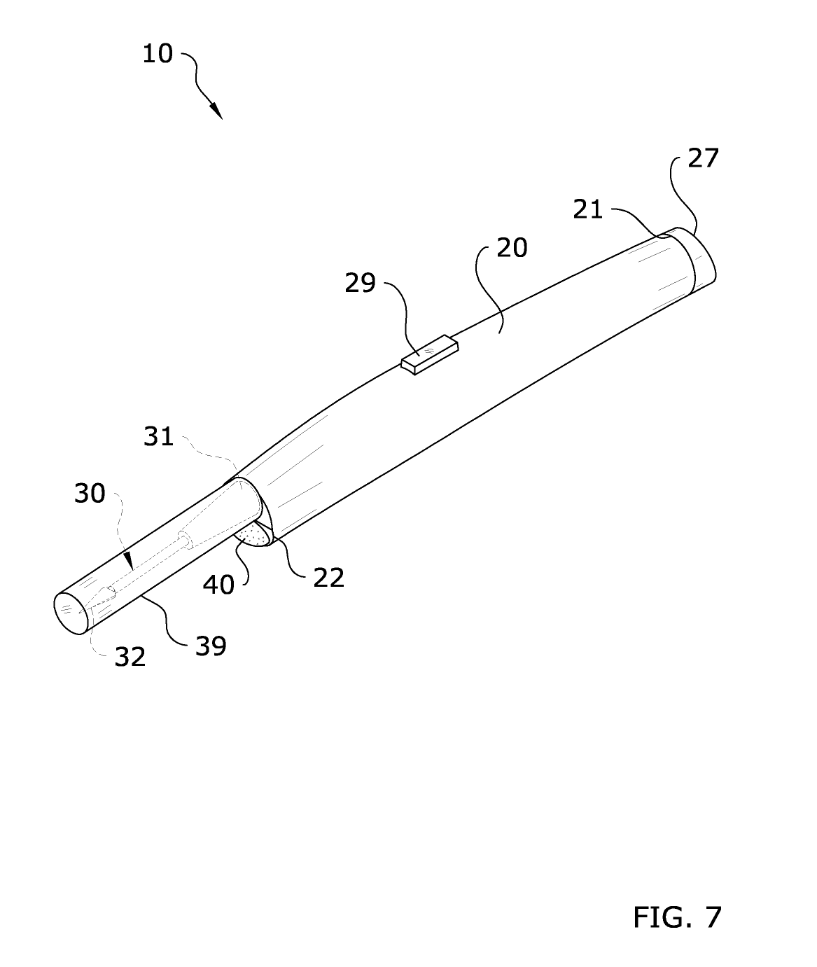

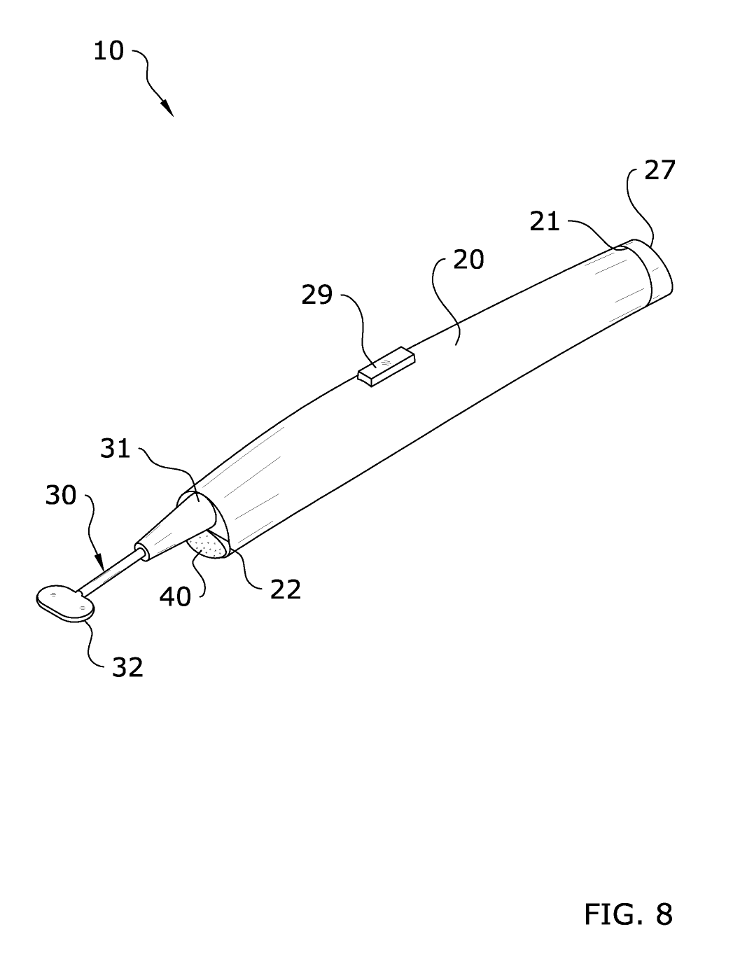

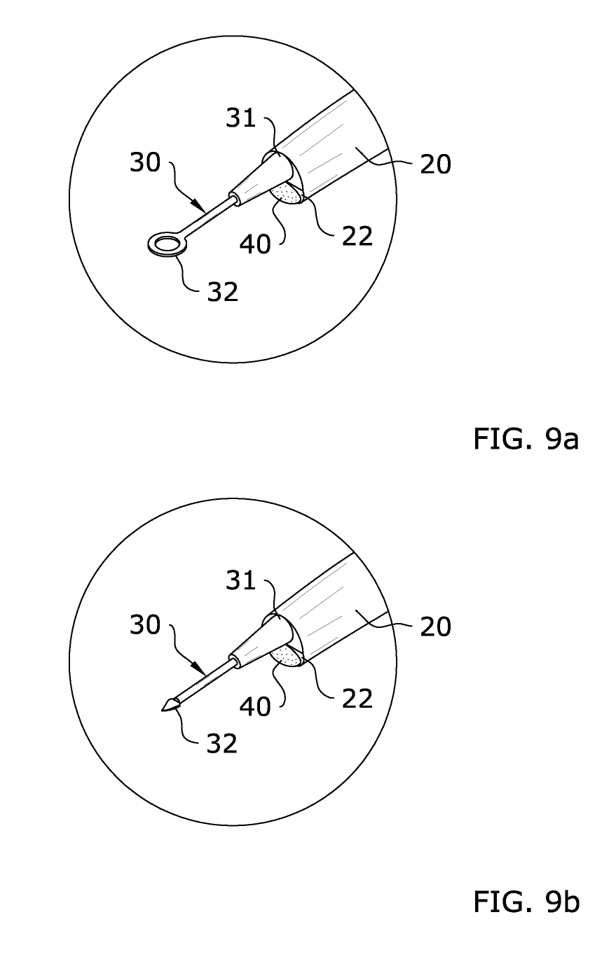

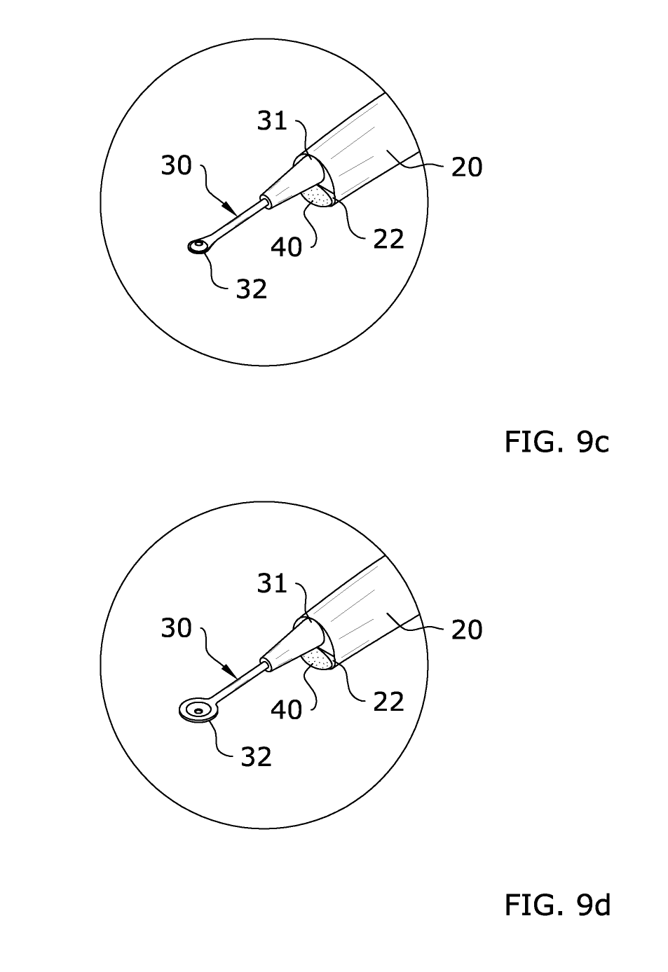

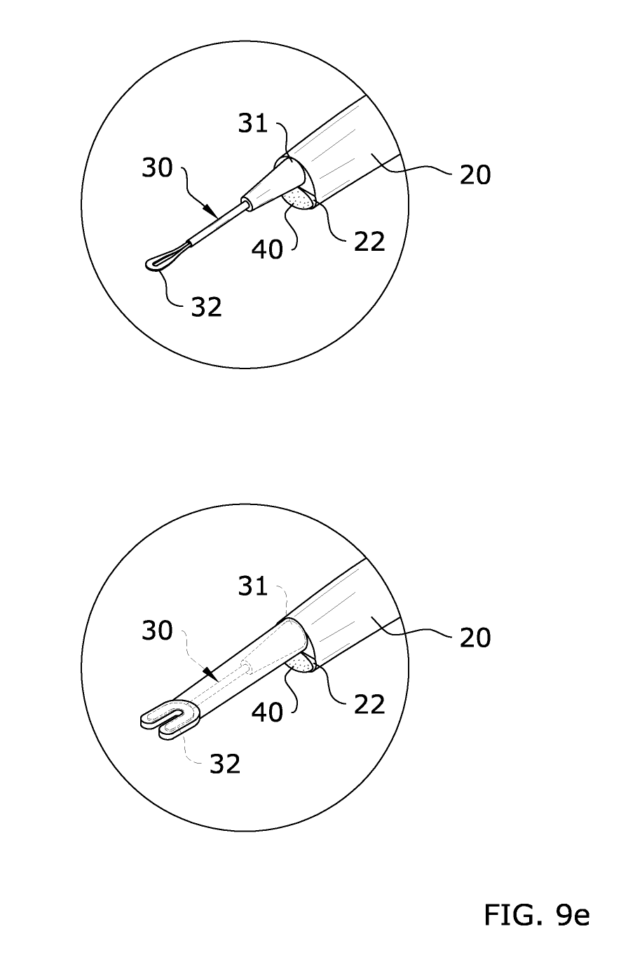

The second end 32 of the extractor 30 may have a looped tip as shown in FIGS. 1-6, a pointed tip as shown in FIG. 7, a flattened tip as shown in FIG. 8, or a wide range of other tip configuration, such as those shown in FIGS. 9a-9e. The present invention should not be construed as being limited to any particular extractor 30 configuration, as any type of extractor 30 tip known in the art to aid in the removal of comedones may be utilized with the present invention.

The extractor holder 35 is comprised of a receiver or other structure which fits within the second end 22 of the housing 20 to receive the extractor 30 therein. The first end 36 of the extractor holder 35 is positioned within the interior portion 28 of the housing 20 in contact with the motor 54. The second end 37 of the extractor holder 35 includes a threaded receiver portion 38 adapted to threadably receive the first end 31 of the extractor 30 therein.

In some embodiments, the extractor 30 may be heated, such as by a heating coil positioned within the housing 20 adjacent to its frontal opening 23. Additionally, as shown in FIGS. 7 and 9e, an extractor cover 39 may be provided in some embodiments to fit over the extractor 30. The cover 39 could be provided to perform a wide range of functions, such as for hygiene purposes where the extractor 30 is to be used on different people.

The shape of the extractor cover 39 may vary along with the different shapes of extractors 30 as shown in the figures. The cover 39 could be disposable or re-usable. The cover 39 could be absorbent, cushioned for comfort, infused with medicine, or adapted to be warmed.

### D. Illumination Device.

The present invention includes an illumination device 40 which aids with illuminating the area from which blemishes are being removed. The illumination device 40 may be positioned at various locations along the housing 20. In a preferred embodiment as shown in the figures, the illumination device 40 is positioned at the front end 22 of the housing 20 adjacent to the extractor 30. Various types of illumination devices 40 may be utilized, such as light emitting diodes, LCD, CFL, halogen, xenon, infrared, blue light, UV, or heated lights.

### E. Power Source and Motor.

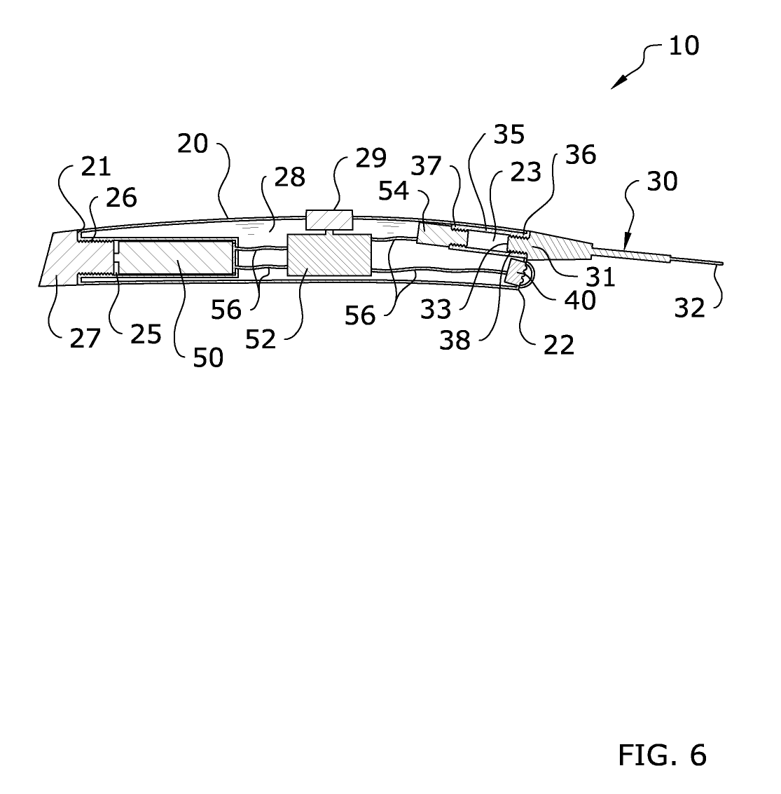

As best shown in FIG. 6, the interior portion 28 of the housing 20 contains a power source 50, circuit board 52, motor 54 and conduits 56. The power source 50 may be comprised of various types of batteries 50, including non-rechargeable and rechargeable varieties. In some embodiments, the power source 50 may be a wired connection to a wall socket.

The circuit board 52 controls activation and deactivation of both the light 40 and the motor 54. The switch 29 is connected directly to the circuit board 52. The circuit board 52 may be adapted to be operated in various manners. In a preferred embodiment, pressing the switch 29 once will turn on the light 40, a second time will turn on the motor 54, and a third time will deactivate both the light 40 and the motor 54. Various other configurations may be utilized. In other embodiments, multiple switches 29 may be utilized (i.e. one for the light 40 and one for the motor 54). In other embodiments, the switch 29 may automatically turn on/off when the extractor 30 touches the user's skin or is removed from a case/charger.

The motor 54 is directly connected with the extractor 30 and/or extractor holder 35 for imparting vibrations to the extractor 30. Preferably, a rotating motor 54 having an offset weight on its drive shaft will be utilized to impart the vibrations to the extractor 30. However, a wide range of motors 54 or other vibrational devices may be utilized to vibrate the extractor 30, including variable speed motors 54. The movement forces imparted to the extractor 30 may be transmitted via vibrations, random movements, oscillations, rotations, reciprocations, and the like. In some embodiments, a constant use spring may be utilized to impart such forces to the extractor 30. In some cases, the present invention may be pneumatically powered.

Electrical conduit 56 is utilized to connect the various components with each other and with the battery 50. As shown in FIG. 6, a first conduit 56 links the power source 50 with the circuit board 52, a second conduit 56 links the circuit board 52 with the motor 54, and a third conduit 52 links the light source 40 with the circuit board 52.

The housing 20 may also incorporate onboard memory, a built-in microcontroller, a built-in timer, or speed adjustors for the motor 54. The microcontroller could be programmed to perform various functions with the present invention, such as controlling motor 54 speed. The on-board memory could be used in combination with the microcontroller to provide updates to its firmware.

### F. Operation of Preferred Embodiment.

In use, the switch 29 is pressed once to activate the light 40. The present invention is then positioned near the blemish to be removed. The switch 29 is pressed again to activate the motor 54, which will cause vibration of the extractor 30. The extractor 30 tip is then placed over or near the blemish and gently pressed down around or near the blemish. The vibration action aids in reducing surface tension of the skin and helps to break loose and dislodge the blemish. The blemish may then be removed along with the tool, and the switch 29 pressed a third time to deactivate both the light 40 and the motor 54 until the tool 10 is used again.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. A comedo extractor, comprising:
a housing including a first end and a second end;
a comedo extractor extending from said second end of said housing;
a motor for vibrating said comedo extractor; and
an illumination device positioned on said housing.

2. The comedo extractor of claim 1, further comprising an extractor holder positioned within said second end of said housing.

3. The comedo extractor of claim 2, wherein said comedo extractor is removably secured within said extractor holder.

4. The comedo extractor of claim 2, wherein said motor is connected to said extractor holder.

5. The comedo extractor of claim 1, further comprising a power source positioned within said first end of said housing.

6. The comedo extractor of claim 1, wherein said illumination device is positioned adjacent to said comedo extractor.

7. The comedo extractor of claim 6, wherein said battery is removably positioned within a battery receiver, wherein said battery receiver is positioned adjacent to said first end of said housing.

8. The comedo extractor of claim 5, further comprising a circuit board connected to said power source by a first conduit.

9. The comedo extractor of claim 8, wherein said circuit board is connected to a switch, wherein said switch is adapted to toggle said motor and said illumination device.

10. The comedo extractor of claim 9, further comprising a second conduit connecting said circuit board with said motor.

11. The comedo extractor of claim 10, further comprising a third conduit connecting said circuit board with said illumination device.

12. A comedo extractor, comprising:
a housing including a first end and a second end;
an extractor holder positioned at said second end of said housing;
a comedo extractor extending from said extractor holder;
a motor for vibrating said comedo extractor;
an illumination device positioned at said second end of said housing adjacent to said comedo extractor; and
a switch for toggling said motor and said illumination device.

13. The comedo extractor of claim 12, wherein said comedo extractor is removably secured within said extractor holder.

14. The comedo extractor of claim 12, wherein said motor is connected to said extractor holder, wherein said motor includes a drive shaft and further comprising an offset weight positioned on said drive shaft.

15. The comedo extractor of claim 12, further comprising a power source positioned within said first end of said housing.

16. The comedo extractor of claim 15, wherein said power source is comprised of a battery.

17. The comedo extractor of claim 12, further comprising a circuit board connected to said power source by a first conduit.

18. The comedo extractor of claim 17, wherein said circuit board is connected to a switch, wherein said switch is adapted to toggle said motor and said illumination device.

19. The comedo extractor of claim 12, wherein said comedo extractor includes a looped tip.

20. A comedo extractor, comprising:
a housing including a first end and a second end;
a battery removably positioned within said first end of said housing;
a circuit board connected to said battery by a first conduit;
an extractor holder positioned at said second end of said housing;
a comedo extractor extending from said extractor holder, wherein said comedo extractor is removably secured within said extractor holder;
an extractor cover removably secured around said comedo extractor, wherein said extractor cover is infused with a medicine;
a motor for vibrating said comedo extractor, wherein said motor is connected to said extractor holder, wherein said motor is comprised of a rotating motor, wherein said motor includes a drive shaft, wherein said drive shaft includes an offset weight, wherein said motor is connected to said circuit board by a second conduit;
an illumination device positioned at said second end of said housing adjacent to said comedo extractor, wherein said illumination device is connected to said circuit board by a third conduit; and
a switch for toggling said motor and said illumination device, wherein said switch is connected to said circuit board.
